(19) United States
(12) Patent Application Publication
(10) Pub. No.: US 2024/0390169 A1
(43) Pub. Date: Nov. 28, 2024

(54) HAND-HELD UNIT FOR FEEDING AND RELEASING AN IMPLANT

(71) Applicant: OPTIMED MEDIZINISCHE INSTRUMENTE GMBH, Ettlingen (DE)

(72) Inventors: Achim ZIPSE, Baden-Baden (DE); Mareike LUTZ, Malsch (DE); Jonas FRANKE, Karlsruhe (DE); Fabian SCHMIDT, Karlsruhe (DE)

(21) Appl. No.: 18/716,923

(22) PCT Filed: Dec. 2, 2022
(86) PCT No.: PCT/EP2022/084192

§ 371 Date: Jun. 5, 2024

(30) Foreign Application Priority Data
Dec. 6, 2021 (DE) 102021132097.6

(51) Int. Cl. A61F 2/95

(52) U.S. Cl. CPC A61F 2/9517

(57) ABSTRACT

A hand-held unit serves to feed an implant, in particular a stent, and to release the implant by means of a release element. The hand-held unit comprises a housing that has a housing opening and a connection section for attaching an outer catheter layer of a catheter to the housing and for guiding the release element from an interior of the housing into the catheter so that the release element is movable relative to the outer catheter layer. The hand-held unit further comprises a transmission element that is connected to the release element in order to transmit a movement of the transmission element to the release element, and an actuation element that is rotatably or displaceably supported in the housing and that can be actuated from outside the housing through the housing opening, wherein a movement of the actuation element causes a movement of the transmission element.

The invention relates to a hand-held unit for feeding and releasing an implant.

Such hand-held units are in particular used to insert an implant, for example a stent, stent graft, covered stent, filter, etc., into the body of a patient and in particular to place it in a blood vessel. In this respect, the implant is first brought to a specific position within the blood vessel by means of a catheter connected to the hand-held unit. Once the correct position for the implant has been reached, the implant is inserted at this position. With self-expanding implants or stents, the insertion can take place by removing a sleeve or a jacket surrounding the implant, also designated as a middle catheter or middle catheter layer, from the implant so that the implant can expand with respect to its periphery, supports the blood vessel and remains in the release position. Self-expanding implants or stents can consist of a shape memory alloy, for example. Other implants or stents can, for example, be expanded by balloon dilatation so that they remain in place.

It is an object of the present invention to improve the operability of a hand-held unit.

This object is satisfied by a hand-held unit having the features of claim 1. Advantageous embodiments are the subject of the dependent claims.

The hand-held unit according to the invention serves to feed an implant, in particular a stent, and to release the implant by means of a release element and comprises a housing that has a housing opening and a connection section. The connection section serves to attach an outer catheter layer of a catheter to the housing and to guide the release element from an interior of the housing into the catheter so that the release element is movable relative to the outer catheter layer.

Furthermore, the hand-held unit has a transmission element that is connected to the release element in order to transmit a movement of the transmission element to the release element, and an actuation element that is rotatably or displaceably supported in the housing and that can be actuated from outside the housing through the housing opening, wherein a movement of the actuation element causes a movement of the transmission element.

According to the invention, the hand-held unit is in particular used to introduce the implant by means of the catheter into a—in particular human—body of a patient, and indeed substantially into a blood vessel of the patient, to position it there and then to release it so that the implant remains at the location at which it was positioned. In this respect, the hand-held unit is held in one hand by the user or practitioner as intended. The catheter in particular has an inner catheter layer, a middle catheter layer and an outer catheter layer, wherein the middle catheter layer is movable relative to the inner and the outer catheter layer and the implant is received between the inner and the middle catheter layer before the release. The term “release” refers to the removal of the implant from the catheter so that the implant can remain at the position in the body or blood vessel of the patient. As a rule, the middle catheter layer surrounding the implant is retracted, i.e. moved away from the implant towards the hand-held unit, while the implant is held radially inside, in particular by a form fit, against the movement of the middle catheter layer on the inner catheter layer. After the retraction of the middle catheter layer, the implant can expand with respect to its periphery. With self-expanding implants, this happens automatically as soon as the middle catheter layer releases the implant. Other, non-self-expanding implants are e.g. expanded by inflating a balloon. According to the invention, the retraction of the middle catheter layer takes place by the movement of the release element transmitted from the transmission element to the release element or by the associated pulling force on the release element. In this respect, the release element usually corresponds to the middle catheter layer, but it is also conceivable that the release element is a separate element that transmits the pulling force to the middle catheter layer.

The actuation element can be actuated, i.e. in particular rotated or displaced, through the housing opening, namely, for example, by the practitioner or user acting on it with one or more fingers. This includes, on the one hand, that the actuation element itself extends from the housing interior through the housing opening so that a part of the actuation element is outside the housing and can be manually moved, in particular rotated or displaced, by a practitioner there. On the other hand, this can also be understood in that the actuation element is indeed arranged completely inside the housing, but a part of the actuation element can be acted on from outside through the housing opening so that the actuation element can be moved.

Such a movement of the actuation element causes a movement of the transmission element that preferably extends parallel to a direction of movement of the release element and/or can be moved parallel to the direction of movement of the release element. The transmission element can in particular enclose the release element. Since the release element is connected to the transmission element, this in turn causes a movement of the release element, whereby the implant is then released. In this respect, the release element can advantageously be moved back and forth in its direction of movement, i.e. it can be repositioned.

The transmission element can, for example, be configured as a rigid or flexible rod or as a chain and can preferably at least partly enclose the release element. A rigid or stiff rod is particularly stable and can be moved back and forth along the direction of movement so that a repositionable system is present. In this respect, the length of the housing of the hand-held unit corresponds at least approximately to the placement path, i.e. the path length over which the release element and the transmission element must be moved to completely place or release the implant. With flexible, non-rigid transmission elements, on the other hand, for example a flexible rod (also called a flex rod) or a chain, the transmission element can be deflected within the hand-held unit by suitable means. In this case, the length of the housing can be selected substantially independently of the placement path.

In an advantageous embodiment, the transmission element is provided with a plurality of engagement sections for the actuation element or for a drive gear. The engagement sections can in particular be arranged at equal spacings from one another at the transmission element, preferably at a side of the transmission element facing the actuation element or the drive gear. For example, the engagement sections can be formed as depressions, in particular notches, recesses or openings, into which teeth or projections of the actuation element or the drive gear can engage to transmit a movement to the transmission element. It is understood that, conversely, the engagement sections can also be configured as projections or teeth, wherein corresponding recesses, depressions or openings are then to be provided at the actuation element or the drive gear.

The transmission element preferably has a plurality of links that adjoin one another and are movable relative to one another, and indeed in particular if the transmission element is configured as a chain. In this case, chain links can also be spoken of. The individual links themselves can be rigid, but they are, for example, joined together or connected by means of (pivot) joints so that a rotary movement of the adjacent links relative to one another is in particular possible at the joints.

A guide element for the transmission element can be arranged in the housing. The guide element can, for example, be tubular with a circular or angular cross-section. The guide element can be completely closed or partly open in cross-section as long as a sufficient guidance function for the transmission element is ensured. When moving the transmission element, the guide element ensures that said transmission element is moved along a predefined movement path so that a tilting or similar interference is avoided within the housing.

The guide element can have a deflection region arranged at one housing end, wherein the guide element and the connection section are preferably arranged at oppositely disposed ends of the housing. Substantially the entire length of the housing, i.e. approximately the spacing between the connection section and the oppositely disposed end, is thereby utilized to move the transmission element and the release element. The guide element can be lifted off the release element in the deflection region. The release element then leaves the guide element, for example, to be guided out of the housing. However, the transmission element, if it is designed as flexible, will be guided further along the guide element through the deflection region.

The guide element extends into the catheter possibly at least before the start of the release of the implant. This corresponds to the treatment situation when the implant has been positioned at the correct position in the body or blood vessel, but the release has not yet been initiated. In other words, the transmission element and the release element have not yet been moved out of their starting position at this time.

The actuation wheel can partly project through the housing opening, which considerably simplifies the accessibility for the practitioner or operator of the hand-held unit, and thus the operability.

The housing can furthermore have a second housing opening, wherein the actuation element can additionally be actuated from outside the housing through the second housing opening. In particular, the actuation element can partly project through the second housing opening. If the actuation element is accessible at two housing openings, it can be manipulated or actuated with several fingers, for example.

The housing openings are preferably arranged at oppositely disposed sides of the housing. It is in particular possible for one housing opening to be arranged at an upper side and the other housing opening at a lower side of the hand-held unit so that one part of the actuation element projects through the one housing opening and another part of the actuation element projects through the other housing opening. The terms “upper side” and “lower side” refer to the housing sides of the hand-held unit that are located at the top or bottom during the intended use. If the housing openings are arranged at oppositely disposed sides, it is possible, for example, to actuate the actuation element simultaneously or alternately with the thumb and index finger of the same hand so that a precise actuation of the actuation element, and thus a precise release of the implant, is possible.

The actuation element can be configured as a rotatably supported actuation wheel. It can then be rotated by the practitioner or user acting on it with one or more fingers. The actuation element or actuation wheel can have a tooth-like contour at its outer periphery so that the contact surface is non-slip and the practitioner or operator does not slip off the actuation wheel when releasing the implant.

In the housing, a rotatably supported drive gear can be provided that is in engagement with the transmission element to cause a movement of the transmission element during a rotary movement of the drive gear. This can in particular take place by means of corresponding torque transmission sections of the actuation wheel and the drive gear that can, for example, be configured as gear wheels and are in engagement with one another. In particular, a reversal of the direction of rotation can take place so that the drive gear rotates in the opposite direction of rotation compared to the actuation wheel. This has the result that the actuation of the actuation wheel can take place conveniently, for example, by the practitioner repeatedly turning the actuation wheel in his direction with his thumb and the release element in so doing being moved in the direction required for releasing or placing the implant. The reversal of the direction of rotation is therefore ergonomically advantageous and thus contributes to a good treatment result.

For example, the torque transmission section of the actuation wheel can project coaxially from a side surface of the actuation wheel and the torque transmission section of the drive gear can project coaxially from a side surface of the drive gear.

In an alternative embodiment, the actuation element can be configured as a displaceably supported sliding knob that preferably has a contact surface for actuation with a finger that can in particular project from the housing. The contact surface can have a textured surface, for example a knurled or ribbed surface, so that the contact surface is non-slip, prevents a slipping off and improves the operability.

The sliding knob is preferably connected to the transmission element. This in particular means that the sliding knob is directly or immediately connected to the transmission element. The connection can be form-fitting, in particular by means of a projection that is provided at the sliding knob and that engages into a depression, a recess or an opening at the transmission element. However, another type of connection would generally also be conceivable, namely a bonded or force-transmitting connection.

In an advantageous embodiment, the housing can have two guide tabs that extend along a direction of movement of the sliding knob and that engage into grooves at oppositely disposed sides of the sliding knob in order to guide the sliding knob so that the movement of the sliding knob is defined by the guide tabs and the grooves.

The housing preferably comprises a guide for guiding the transmission element and/or an inner catheter layer, also called an inner catheter, out of the interior of the housing. A Luer lock connection can be fastened to the end of the inner catheter and to the housing with a form fit. The fastening of the Luer lock connection to the inner catheter can in particular be designed so that a flushing of the inner catheter and an introduction of a guide wire through the inner catheter are made possible. The guide and the connection section are preferably arranged at oppositely disposed ends of the housing.

For better handling, the housing can be provided with depressions for the fingers of an operator or practitioner. The housing then lies particularly comfortably in the hand and can be held securely and steadily during treatment.

The invention will be described schematically and by way of example with reference to embodiments and to the drawings. It is shown therein:

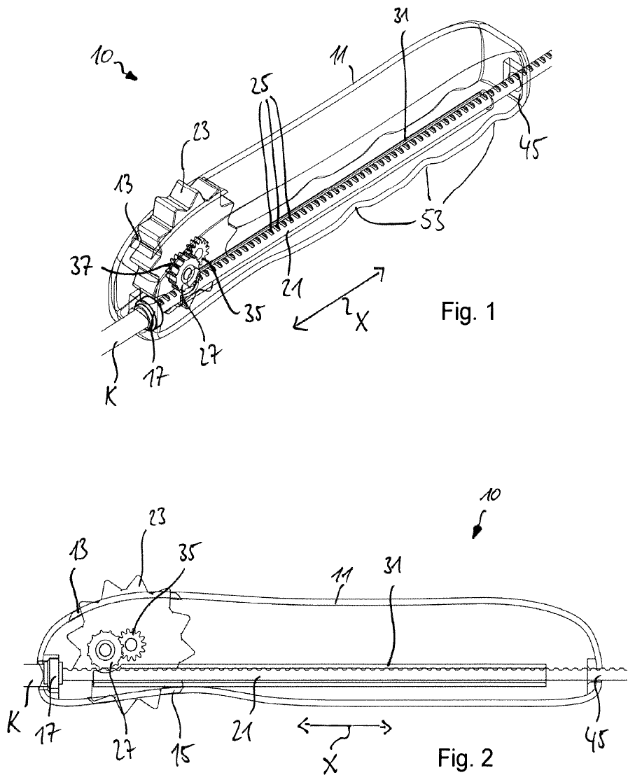

FIG. 1 a perspective view of a hand-held unit according to a first embodiment example;

FIG. 2 a longitudinal sectional view of the hand-held unit of FIG. 1;

FIG. 3 a perspective view of a hand-held unit according to a second embodiment example;

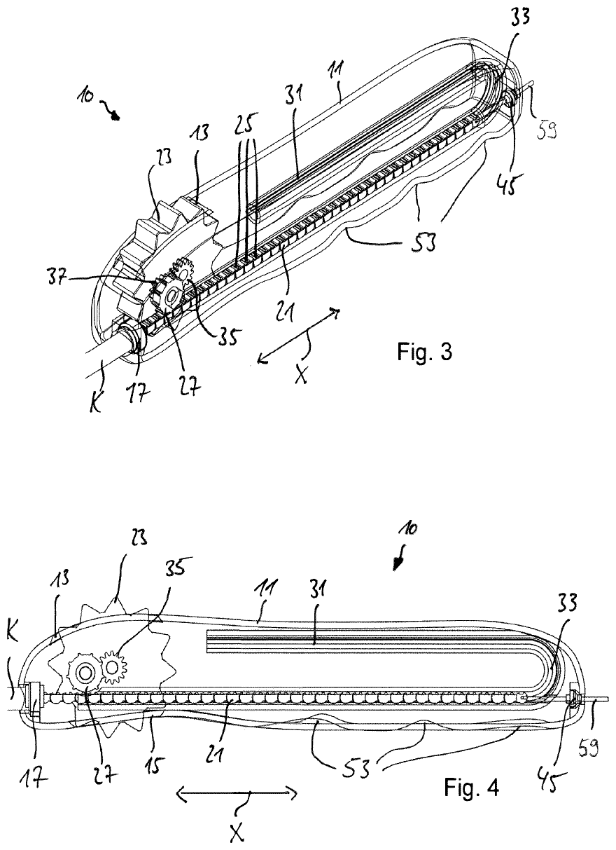

FIG. 4 a longitudinal sectional view of the hand-held unit of FIG. 3;

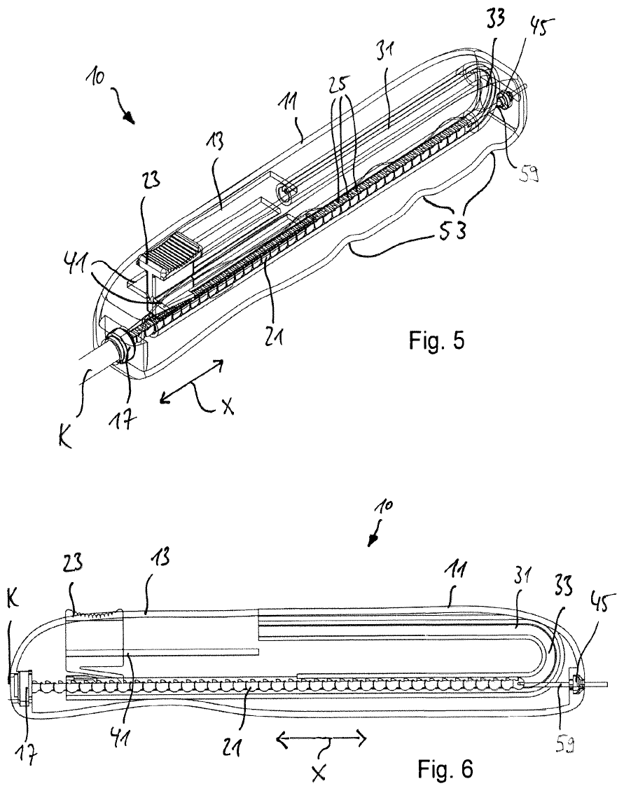

FIG. 5 a perspective view of a hand-held unit according to a third embodiment example;

FIG. 6 a longitudinal sectional view of the hand-held unit of FIG. 5;

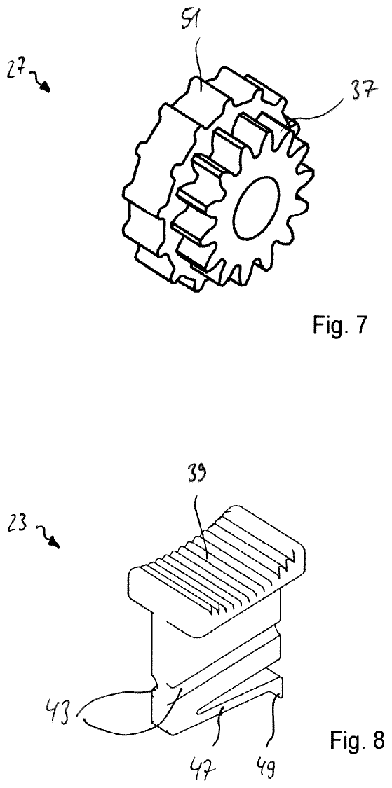

FIG. 7 a perspective view of a drive gear;

FIG. 8 a perspective view of an actuation element configured as a sliding knob;

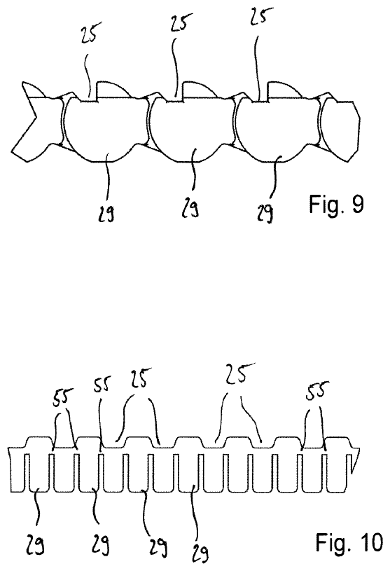

FIG. 9 a side view of a section of a transmission element configured as a chain; and

FIG. 10 a side view of a section of a transmission element configured as a flexible rod.

FIGS. 1 to 6 show three different embodiment examples of a hand-held unit 10. The hand-held unit 10 in each case has an elongate housing 11 whose walls are shown transparent so that the interior of the housing 11 is clearly visible.

The housing 11 has a housing opening 13 and a connection section 17 to which a catheter K is attached with which a stent, not shown, is inserted by means of the hand-held unit 10 into the blood vessel of a patient, is positioned there and is then released to remain in the blood vessel.

A release element 19, which in FIGS. 1 and 2, however, is covered by a transmission element 21 described in more detail later and is therefore not visible, is guided through the connection section 17 out of the housing interior into the catheter K. The release element 19 can be moved back and forth along a direction of movement X relative to the catheter K, in particular to an outer catheter layer and an inner catheter layer. An inner catheter layer extends through the transmission element and is fastened to a proximal end of the housing 11; for this purpose, the transmission element has an opening (not shown). At the proximal end of the housing 11, the transmission element is guided out of the housing 11 by a guide 45. Since the release element 19 can be moved back and forth along the direction of movement X, it is a repositionable system. In the present embodiment example, the release element 19 corresponds to the middle catheter layer that surrounds the stent before the release. The stent is therefore received between the inner and middle catheter layer before the release. Alternatively, it is also conceivable that the release element 19 is formed separately from the middle catheter layer, but can exert a pulling and compressive force on it.

If a force acting on the release element 19 in the housing interior causes a movement of the release element 19 in the direction of movement X away from the outer catheter layer, i.e. towards the guide 45, the release element 19 surrounding the implant is removed from the implant in the catheter K. The implant can thereby expand to remain in the blood vessel.

The force acting on the release element 19 is caused by the aforementioned transmission element 21. The transmission element 21 is connected to the release element 19 for this purpose.

FIGS. 1 and 2 show an embodiment example in which the transmission element 21 is configured as a rigid rod that encloses the release element 19. The transmission element 21 has a plurality of engagement sections 25 that are arranged at equal spacings from one another along an upper side of the transmission element 21 and are formed as depressions or notches made in an outer wall of the transmission element 21.

FIGS. 3 to 6 show two different embodiment examples in which the respective transmission element 21 is configured as a chain, consisting of a plurality of links 29 that adjoin one another and are movable relative to one another. A section of such a chain with a plurality of links 29 is also clearly shown in FIG. 9. The individual links 29 themselves are rigid, but are joined together or connected by means of (pivot) joints (not shown) so that a rotary movement of each link 29 relative to its adjacent link 29 is possible. A recess or opening, which acts as an engagement section 25, is formed in each link 29.

As an alternative to a configuration as a rigid rod or as a chain, FIG. 10 shows a section of a transmission element 21 that is configured as a flexible rod or flex rod. This transmission element 21 also consists of a plurality of links 29 that adjoin one another and are movable relative to one another, but are fixedly connected to one another. This means that the transmission element 21 is formed in one part. The movability of the individual links 29 relative to one another is achieved by a plurality of bridge sections 55 that are arranged along the direction of extent of the transmission element 21 and are formed as very thin perpendicular to the direction of extent so that a certain mobility or flexibility is achieved.

The housings 11 of the different hand-held units 10 in accordance with FIGS. 1 to 6 each have a guide element 31 for the transmission element 21. The guide element 31 is tubular with a circular cross-section in the present embodiment example so that the transmission element 21 is guided within the guide element 31. When moving the transmission element 21, the guide element 31 ensures that said transmission element is moved along a predefined movement path so that a tilting or similar interference is avoided within the housing 11.

In FIGS. 1 and 2, the extent of the guide element 31 is designed as one-dimensional, i.e. the guide element 31 extends in a constant direction since the transmission element 21 is rigid, as described above. At the end of the housing 11, the transmission element 21 is guided out of the housing 11 by the guide 45.

In the two embodiment examples in accordance with FIGS. 3 to 6, in which the transmission element 21 is designed as flexible in each case, the guide element 31 has a deflection region 33, i.e. the guide element 31 is curved and thus changes its direction. The flexible transmission element 21 is then deflected within the guide element 31 and can thus be guided further within the housing 11, for example in the opposite direction. A guiding out of the housing 11 is then not required. To utilize the entire length of the housing 11 for the movement in the guide element 31, the deflection region 33 is arranged at a rear (proximal) housing end. Furthermore, the guide element 31 is lifted off the inner catheter layer 59 in the deflection region 33. This means that the inner catheter layer 59 leaves the guide element 31. In the present embodiment examples, the inner catheter layer 59 is then guided out of the housing 11 by the guide 45. The transmission element 21, on the other hand, is guided further along the guide element 31 through the deflection region 33 so that the housing interior with the guide element 31 then serves as a storage space for the transmission element 21.

The movement of the transmission element 21 is initiated by means of an actuation element 23 in each case. The actuation element 23 can be configured, for example, as an actuation wheel rotatably supported in the housing 11 (see FIGS. 1 to 4) or as a sliding knob displaceably supported in the housing 11 (see FIGS. 5, 6 and 8) and can be actuated from outside by a user or practitioner with one hand due to the housing opening 13. In the present embodiment examples, the actuation element 23 partly projects through the housing opening 13 in each case. In the two embodiment examples in accordance with FIGS. 1 to 4, the housing 11 is also provided with a second (lower) housing opening 15, through which a further part of the actuation element 23 projects, so that the actuation element 23 can be actuated from outside the housing 11 and from the upper side and the lower side of the housing 11 at the same time, for example with the thumb at the upper side and with the index finger of the same hand at the lower side. To further optimize the handling of the hand-held unit 10, it is advisable to provide the housing 11 with depressions 53 at its lower side for the further fingers of the operator so that the housing 11 fits easily in the hand.

The actuation element 23 configured as an actuation wheel has a tooth-like contour at its outer periphery as a contact surface for the fingers of a practitioner or operator so that a practitioner or operator does not slip off the actuation element 23 when releasing an implant. The actuation element 23 configured as a sliding knob, on the other hand, is provided with a contact surface 39 for the fingers of the practitioner or operator that has a textured surface, and indeed a ribbed surface.

In the embodiment examples in accordance with FIGS. 1 to 4, in which the actuation element 23 is configured as an actuation wheel, a drive gear 27, which is shown separately in FIG. 7, is further supported in the housing 11. The actuation element 23 and the drive gear 27 are then each provided with a torque transmission section 35, 37. The torque transmission sections 35, 37 are configured as gear wheels that, in the present example, are formed in one part with the actuation element 23 or the drive gear 27 and project coaxially from respective side surfaces of the actuation element 23 or the drive gear 27. The torque transmission sections 35, 37 are in engagement with one another, and indeed such that a rotation of the actuation element 23 is transmitted to the drive gear 27 with a reversal of the direction of rotation. A toothing 51 at the outer periphery of the drive gear 27 engages into the engagement sections 25 of the transmission element 21 and thus converts the rotary movement of the drive gear 27—apart from the deflection in the deflection region—into a linear, one-dimensional movement of the transmission element 21 and thus of the release element 19.

As FIGS. 5 and 6 show, the actuation element 23 configured as a sliding knob, on the other hand, engages directly into the engagement sections 25 of the transmission element 21, namely by means of a projection 49 that projects from a lower, inclined web 47. A form fit effective in the direction of movement X is achieved by the projection 49 engaging into the engagement sections 25. The actuation element 23 configured as a sliding knob is furthermore provided with grooves 43 which are arranged at oppositely disposed sides of the sliding knob and into which guide tabs 41 engage in order to achieve a linear guidance of the actuation element 23 at the housing 11, in particular along the housing opening 13. When the actuation element 23 is moved along the linear guide, this movement is transmitted directly to the transmission element 21, and thus to the release element 19, due to the above-described form fit. Thus, when the actuation element 23 is actuated, an implant can be released in a blood vessel due to the transmission of the movement to the transmission element 21 and the release element 19.

### REFERENCE NUMERAL LIST

10 hand-held unit
11 housing
13 housing opening
15 housing opening
17 connection section
19 release element
21 transmission element
23 actuation element
25 engagement section
27 drive gear
29 link
31 guide element
33 deflection region
35 torque transmission section
37 torque transmission section
39 contact surface
41 guide tab
43 groove
45 guide
47 web
49 projection
51 toothing
53 depression
55 bridge section
59 internal catheter layer
K catheter
X direction of movement

1-16. (canceled)

17. A hand-held unit for feeding an implant and for releasing the implant by means of a release element, said hand-held unit comprising
a housing that has a housing opening and a connection section for attaching an outer catheter layer of a catheter to the housing and for guiding the release element from an interior of the housing into the catheter so that the release element is movable relative to the outer catheter layer,
a transmission element that is connected to the release element in order to transmit a movement of the transmission element to the release element, and
an actuation element that is rotatably or displaceably supported in the housing and that can be actuated from outside the housing through the housing opening, wherein a movement of the actuation element causes a movement of the transmission element.

18. The hand-held unit according to claim 17,
wherein the transmission element is configured as a rigid or flexible rod or as a chain.

19. The hand-held unit according to claim 17,
wherein the transmission element is provided with a plurality of engagement sections for the actuation element or for a drive gear.

20. The hand-held unit according to claim 17,
wherein the transmission element has a plurality of links that adjoin one another and are movable relative to one another.

21. The hand-held unit according to claim 17,
further comprising a guide element, which is arranged in the housing, for the transmission element.

22. The hand-held unit according to claim 21,
wherein the guide element has a deflection region arranged at one housing end.

23. The hand-held unit according to claim 17,
wherein the transmission element extends into the catheter at least before the start of the release of the implant.

24. The hand-held unit according to claim 17,
wherein the actuation element partly projects through the housing opening.

25. The hand-held unit according to claim 17,
wherein the housing has a second housing opening, wherein the actuation element can additionally be actuated from outside the housing through the second housing opening.

26. The hand-held unit according to claim 25,
wherein the housing openings are arranged at oppositely disposed sides of the housing.

27. The hand-held unit according to claim 17,
wherein the actuation element is configured as a rotatably supported actuation wheel.

28. The hand-held unit according to claim 27,
further comprising a drive gear that is rotatably supported in the housing and that is in engagement with the transmission element to cause a movement of the transmission element during a rotary movement of the drive gear, wherein the actuation wheel and the drive gear have corresponding torque transmission sections that are in engagement with one another.

29. The hand-held unit according to claim 17,
wherein the actuation element is configured as a displaceably supported sliding knob.

30. The hand-held unit according to claim 29,
wherein the sliding knob is connected to the transmission element.

31. The hand-held unit according to claim 29,
wherein the housing has two guide tabs that extend along a direction of movement of the sliding knob and that engage into grooves at oppositely disposed sides of the sliding knob in order to guide the sliding knob.

32. The hand-held unit according to claim 17,
wherein the housing comprises a guide for guiding an inner catheter layer and/or the transmission element out of the interior of the housing.

33. The hand-held unit according to claim 22,
wherein the guide element and the connection section are arranged at oppositely disposed ends of the housing.

34. The hand-held unit according to claim 25,
wherein the actuation element partly projects through the second housing opening.

35. The hand-held unit according to claim 27,
wherein the actuation wheel has a tooth-like contour at its outer periphery.

36. The hand-held unit according to claim 29,
wherein the sliding knob has a contact surface for actuation with a finger.

37. The hand-held unit according to claim 36,
wherein the contact surface has a textured surface.

38. The hand-held unit according to claim 37,
wherein the textured surface is one of a knurled surface and a corrugated surface.

39. The hand-held unit according to claim 30,
wherein the sliding knob is connected to the transmission element in a form-fitting manner.

40. The hand-held unit according to claim 32,
wherein the guide and the connection section are arranged at oppositely disposed ends of the housing.

41. The hand-held unit according to claim 18,
wherein the transmission element encloses the release element.
